(19) United States
(12) Patent Application Publication
(10) Pub. No.: US 2015/0320838 A1
(43) Pub. Date: Nov. 12, 2015

(54) COMPOSITION AND FORMULATION FOR TOPICAL TREATMENT OF INFLAMMATION AND TISSUE DAMAGE

(71) Applicant: Giellepi S.p.A., Milano MI (IT)

(72) Inventors: Carlo TERRUZZI, Briosco MB (IT); Luca DEGRASSI, Udine UD (IT)

(21) Appl. No.: 14/409,130

(22) PCT Filed: Jun. 20, 2013
(86) PCT No.: PCT/IB2013/055066

§ 371 Date: Dec. 18, 2014

(30) Foreign Application Priority Data
Jun. 20, 2012 (IT) MI2012A001076

(51) Int. Cl. A61K 38/38; A61K 45/06; A61K 9/70; A61K 9/00

(52) U.S. Cl. CPC A61K 38/38; A61K 9/0014; A61K 45/06; A61K 9/7023

(57) ABSTRACT

The present invention relates to a composition for use in the treatment by topical administration of inflammatory diseases associated with tissue degeneration and/or tissue damage in humans or animals, comprising dehydrated avian egg albumen.

The present invention relates to a composition and a formulation for topical use in the treatment of inflammatory diseases associated with tissue degeneration.

It is known that all inflammatory diseases are associated with degeneration and/or tissue damage. In particular, degeneration and/or tissue damage is associated with each acute inflammation (abscesses, trauma, strains), and any chronic inflammation such as osteoarthritis, autoimmune diseases such as rheumatoid arthritis, inflammatory processes of viral etiology, eczema, erythema, psoriasis etc.

Clinically, an inflammatory process is characterized by symptoms such as pain, heat at the inflamed site, redness, swelling and inhibition or impairment of function. These signs are the manifestations of the above mentioned elemental phenomena of inflammatory response.

Multiple diseases are manifested by symptoms which is the result of an inflammatory process.

Currently the treatment of inflammation has essentially two objectives, namely to remove, if possible, the cause of the inflammation and to decrease the symptoms of inflammation mentioned above.

For this second objective steroidal and non-steroidal anti-inflammatory drugs (NSAIDs) are commonly used.

However, both steroidal and non-steroidal drugs have several known undesired side effects.

In recent years anti-inflammatory therapies based on physical means, for example, TENS therapies (Transcutaneous Electrical Nerve Stimulation), Laser and radiation therapies with light of appropriate wavelength, TECAR therapy (Resistive capacitive energy transfer), and treatment with ozone have been widely used.

More recently, “biotechnological” drugs, consisting of new molecules that interact with newly discovered chemical mediators, appeared on the market.

The results of the above-mentioned anti-inflammatory therapies are not always satisfactory. In addition, the side effects of drugs and other therapeutic approaches represent a strong limit and may require temporary or permanent suspension of therapy.

It is also known from RU2191551 a method for the treatment of simple inflammation of the frontal sinuses, which comprises the injection of diluted egg white.

FR2810550 describes a solution for ocular and nasal washes for human and animal use comprising an homogeneous extract of white and red of quail egg. No cell regeneration effect of this solution is specifically mentioned.

WO97/35595 describes an anti-inflammatory composition obtained from avian eggs that has been hyperimmunized with one or more antigens, a method of isolation of the anti-inflammatory composition and a method of treatment of infections by the administration of the composition, wherein the oral, parenteral, rectal, subcutaneous, intravenous and intranasal routes of administration are mentioned.

WO00/43020 describes an anti-inflammatory composition obtained from purified fractions of yolk and albumen of hyperimmunized avian eggs, and the use of such a composition for the treatment of inflammation, in particular arthritis. Oral administration of such a composition in animal models of collagen-induced arthritis is also described. However, a lack of effectiveness of the anti-inflammatory compositions obtained from egg white is highlighted. No examples of topical administration of such anti-inflammatory composition are described.

Adam M: “Welche Wirkung Gelatinepraparate haben?; Therapie der Osteoarthrose” describes a treatment of osteoarthritis by oral administration of hen egg white. The author concludes that the treatment does not provide significant results. No topical treatment with egg whites or its derivatives is mentioned.

The article “Requirements for egg processors” pages 1-7 published on the internet http://www.foodauthority.nsw.gov.au/_Documents/industry_pdf/egg_processors.pdf describes liquid and dehydrated egg white.

Speciani, Attilio; Piuri Gabriel: “Arthritis and Osteoarthritis,” published on the internet http://eurosalus.com/malattie/malattia/disturbi-artro-reumatici 3 Aug. 2012, page 4, states that according to folk medicine, whipped egg white to be applied locally, or a hot omelet to be left in place for at least half an hour can be used for acute back pain.

However, tests carried out by the inventors have revealed the ineffectiveness of this treatment. Moreover, because of the gelatinous consistency and the tertiary structure of the protein component, egg white cannot be effectively used as such for topical application for the purposes described in the present invention. Therefore, besides being ineffective, albumen as such has proven to be difficult in the application.

Furthermore, the perishability of albumen is not compatible with the requirements of stability and durability of a composition for therapeutic use.

Purpose of the present invention is therefore to provide a composition for the treatment of degenerative diseases and/or tissue damage and both acute and chronic inflammation related thereto, that is free from the aforesaid drawbacks of the known art.

This object is achieved with a composition whose main features are specified in claim 1, a pharmaceutical formulation whose characteristics are specified in claim 14, a patch whose characteristics are specified in claim 17 and a spray whose characteristics are specified in claim 18. Other features of the composition and the formulation according to the present invention are specified in the remaining claims.

It has in fact been surprisingly discovered that topical application of a composition comprising dehydrated egg white stimulates a potent activity of tissue regeneration, in other words it induces the processes for the regeneration of healthy tissue following topical administration.

Moreover, it was further discovered that said composition comprising dehydrated egg white has a significant anti-inflammatory activity following topical administration.

Finally, it was surprisingly found that these anti-inflammatory and regenerative activities are dose-dependent.

The topical application of the active ingredients allows to reduce significantly all the systemic side effects related to the enteral or parenteral administration.

In the present description and in the claims, topical use or topical application means a direct application on the skin surface of man or animal.

An advantage of the composition and the formulation according to the present invention lies in its ease of application associated with a high efficacy. In fact, the composition comprises dehydrated egg white in a form that makes it effectively suitable for topical application in the treatment of inflammatory diseases and degeneration and/or tissue damage associated with them.

Such degeneration and/or tissue damage may not be clinically evident.

The composition and the formulation according to the present invention are also effective in the treatment of inflammatory diseases that are not associated with any evident symptoms of degeneration or tissue damage.

A further advantage of the composition and the formulation according to the present invention consists in the flexibility of use and ease of storage.

Furthermore, the composition and the formulation according to the present invention have the advantage of comprising a physiologically compatible active substance which is therefore devoid of side effects, particularly in the topical use. The absence of side effects, combined with the highly effective anti-inflammatory and unexpected regenerative properties, are the prerequisites for use on a wide scale of the composition according to the present invention also in pathologies that do not have sufficient benefit, or even no benefit at all from currently available drugs.

Surprisingly, the tissue regeneration and anti-inflammatory properties of the composition and formulation for topical use according to the present invention are apparent both at the level of the surface layers, such as the skin and skin appendages, and at the level of deep tissues, such as the connective tissue, muscle tissues, joints, ligaments and synovial.

Furthermore, the anti-inflammatory effect of the composition and formulation for topical use according to the present invention is surprisingly independent of the etiology of inflammation, and produces the effects of marked decrease in the swelling or edema, marked reduction of pain, reduction or disappearance of skin redness, where present, and disappearance of fibrocicatritial reactive lesions, where present, in addition to a marked recovery of function.

Regenerative activity, in the present description and in the claims, refers to the set of processes that lead to the complete repair of a damaged tissue for direct or indirect stimulation of self-reparative mechanisms of the organism itself and subsequent regeneration of the completely healthy, intact and functionally effective tissue of origin. It is well known that the outcome of an acute inflammation can lead to partial regeneration with formation of granulation tissue also said cicatritial tissue; cicatritial tissue itself is the potential source of a chronic inflammation for the loss of the characteristics of the original tissue. An example is the post-traumatic cicatritial fibrosis of muscles and ligaments that is in itself a functional limitation and may, in certain districts, be the cause of the onset of a chronic inflammation which in the time turns into a worsening degenerative disease.

The beneficial effect of the composition and the formulation according to the invention are conducted against all kinds of inflammatory diseases. Inflammatory diseases, in the present description and in the claims, indicates any phenomenon of protective response resulting in the harmful action of physical, chemical and biological agents characterized by symptoms such as pain, heating of the inflamed part, redness, swelling and impediment or functional alteration.

The composition and the formulation according to the present invention have resulted particularly effective for use in the topical treatment of chronic inflammatory pathology, chronic degenerative inflammatory pathology, of chronic osteoarthritis, acute inflammatory pathology, abscesses, mastitis, of articular osteoarthritis pathologies, inflammatory pathology of tendons and/or ligaments, inflammation and tissue damage resulting from autoimmune pathologies, rheumatoid arthritis, inflammatory pathology of viral or fungal etiology and inflammatory pathology and/or tissue damage of the skin, especially eczema and psoriasis, in the treatment of parenchymatous tissues inflammations, in the treatment of degenerative diseases of parenchymatous tissues, in inflammatory pathology of viral etiology, in inflammatory pathology of fungal etiology and in the treatment of tissue degeneration after effort and/or for functional recovery after effort.

For example, by way of illustration, and not by way of limitation, this definition includes chronic inflammatory pathology of the joints, acute inflammatory pathology of the joints, of the muscle-ligament tissue and of the connective tissue, abscesses, mastitis and the superficial skin inflammations.

Among the chronic inflammatory pathology of the joints may be mentioned recurrent arthritis, inflammatory pathologies such as degenerative arthrosis, inflammatory autoimmune pathology such as rheumatoid arthritis, inflammatory metabolic diseases such as diabetes and gout, and polyarteritis nodosa.

Among the acute inflammatory pathology, such as those of the joints and of the muscle-ligament tissue, may be mentioned tendonitis of the tissues of the arm, of the wrist or of the hand, tennis or golfer elbow, tendinitis of the tissues of the legs, of the ankle or of the foot, abscesses, for example subareolar abscesses or mastitis. In the latter pathology, which can occur during breastfeeding, a therapeutic approach is crucial to solve the problem of the mother without compromising the proper breastfeeding with breast milk, which should be suspended in case of systemic medications assumption.

Additional forms of acute inflammation are represented by the inflammatory response that is observed after a surgery, for example after surgery to the ligaments of the knee, of the ankle or of the shoulder, or by the inflammation that is observed after a course of radiotherapy in oncology. Finally, it can be mentioned the inflammation of the skin and annexes after hair removal with wax or adhesive strips.

The composition and the formulation according to the present invention also have a beneficial effect from the aesthetic point of view, as they promote the complete regeneration of damaged tissues. Therefore, according to a particular aspect, the invention relates to a composition and a formulation for cosmetic purposes and/or for use in the topical treatment of cellulite.

The composition according to the present invention, usable for topical skin use in the treatment of all inflammatory diseases, comprises avian egg dehydrated albumen or an extract or derivative thereof.

With dehydrated albumen extract, in the present description and in the claims, it is intended to indicate a substance isolated from the dehydrated albumen.

With dehydrated albumen derivative, in the present description and in the claims, it is intended to indicate a substance isolated from the dehydrated albumen and treated to improve its capacity for permeation through the skin.

Preferably, as dehydrated albumen in the composition according to the present invention it is used dehydrated albumen of a hen egg.

The dehydration of said albumen is preferably carried out by techniques known in the food industry, for example by spray-drying (spray-dry or fluid bed) or lyophilization, dry pasteurization.

The composition according to the present invention therefore comprises the proteins present in egg albumen, for example ovalbumin, ovotransferrin, ovomucoid, ovoglobulin, ovomucin, lysozyme, ovoinhibitor, ovoglycoprotein, flavoprotein, macroglobulin, avidin and cystatin that are formulated according to one of the preferred compositions of the invention.

According to one of these preferred embodiments of the invention, the composition for topical anti-inflammatory use comprises an amount of lysozyme comprised between 1% and 20%, preferably between 5% and 20% with respect to the dry weight of the composition.

The composition according to an alternative embodiment of the invention further comprises NaCl.

The composition according to an alternative embodiment of the invention comprises one or more other molecules having complementary and/or synergistic activity such as antibiotics, antifungals, antioxidants, steroidal and non-steroidal anti-inflammatories, myorelaxants, molecules with proteolytic activity, molecules with anticoagulant properties, biotech drugs, natural origin drugs.

For example, as antioxidants, the composition according to the present invention may comprise polyphenolic antioxidants, such as for example trans-resveratrol; bioflavonoids; xanthophylls such as astaxanthin, zeaxanthin and lutein; lipoic acid and its salts; curcuma and its derivatives such as for example BCM-95® extract; ubiquinones, such as for example coenzyme Q10; vitamin E; DL-phosphoserine.

As steroidal anti-inflammatories, the composition object of the present invention may preferably comprise cortisone, prednisolone, methyl prednisolone, triamcinolone, mometasone, budesonide, betamethasone, dexamethasone, hydrocortisone and/or their salts or complexes.

Non-steroidal anti-inflammatories preferred in the composition object of the present invention are aspirin, nimesulide, piroxicam, ketoprofen, diclofenac, ibuprofen, paracetamol, cyclosporin, methotrexate.

Thiocolchicoside can be for example used as a myorelaxant.

Hyaluronidase can be for example used as a proteolytic molecule.

Heparin can be for example used as anticoagulant.

As biotechnological drugs, the composition according to the present invention may comprise for example TNF-antagonists such as for example efalizumab, abatacept, infliximab, and other molecules that interfere with other components potentially involved in the inflammatory process such as for example ustekinumab (anti IL-12 and IL-23), trastuzumab (anti HER2/neu), abatacept (anti CTLA-4).

Among the natural origin drugs may be mentioned for example arnica montana extract, menthol, camphor and capsicum.

According to one embodiment of the invention, the above described composition for topical anti-inflammatory use is in the form of a powder, to be suspended in water by the final user in order to allow the application on the area affected by degeneration or tissue damage and/or inflammation.

According to a further aspect thereof, the invention relates to a pharmaceutical formulation for skin topical use comprising the above described composition, which is formulated in one of the following forms suitable for topical application to the skin: cream, gel, ointment, spray, medicated gauze, oil in water emulsion or water in oil emulsion, suspension, poultice, medicated implant, micro-emulsion and nano-emulsion, two-phase micellar gel, bath foam, medicated patch or pre-impregnated patch, lotion.

The pharmaceutical formulation according to the present invention comprises a composition according to the invention and at least one excipient suitable for topical use.

The formulation according to the present invention may further comprise antimicrobials, antioxidants, stabilizers, emulsifiers, thickeners, substances that increase the absorption, flavorings and other substances approved for pharmaceutical, nutritional or cosmetic use according to the type of desired formulation.

Excipients suitable for the realization of topical forms such as for example creams or emulsions are, for example, monoglycerides and diglycerides of fatty acids, medium chain triglycerides, saturated fatty acids, linear aliphatic hydrocarbons, polysorbates, sorbitans, anionic, cationic and amphoteric surfactants, polysiloxanes, starches, alginates, gums, oils and fats of animal or vegetable origin, for example shea butter, cellulose derivatives, carboxyvinyl polymers, carboxyvinyl alkylated polymers, acrylic polymers, acrylic polymers esterified with glycerin, oils, silicones, ethers, polyalcohols.

Preferably, a cream or ointment formulation according to the present invention comprises at least one excipient selected from the group consisting of shea butter, ethylene glycol, diethylene glycol, triethylene glycol, propylene glycol, glycerin, petrolatum, sodium alginate and linear hydrocarbon derivatives.

A formulation according to one embodiment of the present invention comprises dehydrated checken egg albumen, propylene glycol, pentaerythritol tetraisostearate and hectorite clay.

A formulation according to another embodiment of the present invention comprises dehydrated avian egg albumen and linear hydrocarbon derivatives, preferably petrolatum, present in an amount comprised between 1% and 75% by weight, relative to the total weight of the two components dehydrated egg albumen and petrolatum.

According to a particularly preferred embodiment of the invention, there is provided a cream comprising dehydrated hen egg albumen in an amount comprised between 20% and 40% by weight, more preferably between 25% and 35%, petrolatum in an amount comprised between 60% and 80% by weight, more preferably between 65% and 75%, and vitamin E acetate in an amount comprised between 0.1% and 1% by weight, more preferably between 0.3% and 0.5%.

Another formulation according to the present invention comprises dehydrated avian egg albumen and aliphatic hydrocarbons in an amount comprised between 10% and 90% by weight with respect to the total weight of dehydrated egg albumen. Preferably, said aliphatic hydrocarbons are selected from propene, butane and butene. This formulation may comprise propylene glycol, wherein the amount by weight of said propylene glycol is comprised between 1% and 60% by weight with respect to the total weight of the two components dehydrated albumen and propylene glycol. More preferably, the amount by weight of said propylene glycol is comprised between 5% and 45% by weight with respect to the total weight of the two components.

According to a particular aspect of the invention, there is provided a pressurized container or a spray bottle containing a formulation as described above comprising at least one pressurized aliphatic hydrocarbon. In this way, the administration of the formulation according to the present invention is performed in a particularly comfortable and clean way thanks to said aliphatic hydrocarbon under pressure, which has the purpose of spreading the contents of the bottle in the form of spray.

The pressurization, therefore, has the sole purpose of enabling the supply of the formulation of the spray can and has no influence on the therapeutic effectiveness of the formulation.

According to another aspect of the invention, there is provided a patch that is medicated or pre-impregnated with a formulation as described above, comprising a composition according to claim 1 and at least one excipient suitable for formulations for topical use. Preferably, the patch according to the present invention comprises a support in non-woven fabric impregnated with a suspension comprising dehydrated egg albumen and alginate gel. Alginate gel means an aqueous solution of sodium alginate with a concentration in weight comprised between 8% and 16%. The suspension may possibly include other components, for example glycerin. The amount of dehydrated egg albumen in said suspension is preferably comprised between 20% and 60%, more preferably between 30% and 50%. Preferably, the support in non-woven fabric is coated with the suspension in a quantity such as to result in a final concentration of dehydrated egg albumen comprised between 6 mg/cm² and 30 mg/cm² of the support. After suspension drying, the steps of reeling of the coated, cutting the reel in strips, die cutting and packaging as single-dose patch are performed. The thus obtained patch is hydrated before use, to activate the adhesive properties of alginate gel that allow a perfect adhesion to the skin.

Another preferred formulation according to the present invention includes dehydrated avian egg albumen and a suitable solvent medium, such as water. Preferably, this formulation comprises dehydrated avian egg albumen in an amount comprised from 5% to 40% by weight and from 60% to 95% by weight of water. More preferably, the formulation according to the present invention comprises from 20% to 30% by weight of dehydrated avian egg albumen and from 70% to 80% by weight of water.

The inventors have found that the anti-inflammatory activity of the composition and of the formulations according to the invention are dose dependent. In particular, it has been compared the anti-inflammatory effect of an egg albumen simply subjected to mechanical straightening of the proteins, but not dehydrated, with the effect of a formulation according to one of the preferred embodiments of the present invention. The clinical results obtained in patients suffering from chronic knee arthropathy, subjected to treatment, indicate that the major concentration of the formulations according to the invention induces a more marked anti-inflammatory effect.

The mode of use of the composition and the formulation according to the present invention depend on several factors: the area to be treated, the general conditions of the subject, the type of pathology and/or etiology underlying the inflammation.

The composition and/or the formulation according to the present invention are preferably suitable for a topical treatment with a duration of at least fifteen minutes, more preferably of at least thirty minutes, still more preferably of at least six hours. In other words, the composition and/or the formulation according to the present invention are in a form such as to allow the maintenance of the active ingredient on the skin overlying the part affected by tissue damage or by the inflammatory process for a period of at least fifteen minutes, more preferably of at least thirty minutes, still more preferably of at least six hours. To maintain in situ the composition and/or the formulation and depending on the preferred embodiment, a support can be used, for example a gauze, a portion of non-woven fabric or absorbent paper, taking care that the support always maintains a sufficient quantity of the formulation according to the present invention on the skin.

The composition and/or the formulation according to the present invention are further suitable for topical application through appropriate garments, which then allow the contact between the formulation and the skin. These garments are appropriately pretreated with the composition or the formulation according to the present invention. For example, such garments may be impregnated with the formulation or the composition according to the present invention or microcapsules may be deposited thereon for the controlled release of said composition or formulation, according to the technologies known in the field. As an example of garments that can be pretreated, elbow pads, knee pads, girdles, stocks, and other garments such as underwear or sport garments may be mentioned. These garments are therefore suitable for night, day, temporary, localized treatments, for sport and/or recreational use and/or in the medical, human and animal cosmetic field.

Typically, and for ease of application, the formulation can be applied in the evening and maintained at the site of application by using a flexible impermeable film, for example, a patch or a shrink film. The following morning, the compress may be removed and the skin may be washed and dried.

The duration of the single application is then typically 6-10 hours, preferably overnight.

Applications of the composition and the formulation according to the present invention of even shorter duration, of at least fifteen minutes, have been nevertheless sufficient to show the anti-inflammatory and tissue regeneration effects.

In the case of inflammation by acute trauma, for example a joint trauma or also in the case of inflammation after a surgical operation, or also in the treatment of abscesses and mastitis, the anti-inflammatory effect, measurable in terms of recovery time reduction and in symptoms improvement (pain, swelling, etc.), is shown even after the first application. Obviously, the improvement of symptoms varies depending on the severity of tissue damage and on its repairing process necessary for recovery of the healthy tissue.

The composition and the formulation according to the invention have wide application in all inflammatory processes in which it is appropriate to contain the inflammatory reaction and simultaneously to stimulate the tissue regeneration: the treatment of inflammatory processes subsequent to physical damage (such as radiotherapy, hair removal etc.), chemical damage (such as burns caused by acid substances etc.) or biological damage (such as infection e.g. acne, abscesses and mastitis, autoimmune diseases e.g. rheumatoid arthritis, Crohn's disease etc.) therefore are comprised in these applications.

The composition and the formulation according to the invention may be used to promote tissue regeneration even in the absence of inflammation, for example in the recovery and regeneration of muscle tissue after physical effort or for prolonged muscular degenerative processes due to situations of particular stress or intense sports training. Further examples of degenerative processes and/or tissue damage without inflammation are represented by protracted microtraumas and degeneration and/or tissue damage consequent to hypoxia by prolonged lack of blood supply.

It is important to point out that, compared to other therapeutic approaches, the composition and the formulation according to the present invention have a high efficacy and promote an optimal recovery of the damaged tissue with a regeneration of the original tissue.

In the case of chronic diseases (eg osteoarthritis) the anti-inflammatory effect of a cycle of treatment of 7 days involves the remission of the inflammatory symptoms for a period of about 3-5 weeks. After this period a further 7-days cycle of application shows again the anti-inflammatory effect that was observed at the beginning of the first cycle of treatment.

Therapy application cycles of 7 days each, every 3-5 weeks for a long time, for example for several years, showed a gradual and marked functional recovery and a lower aggressiveness of phlogistic relapses, compared to that obtained with other remedies or principals previously tested during the current medical therapy. This observation demonstrates for the first time that the composition according to the invention, in addition to the remarkable anti-inflammatory effect mentioned above, has a surprising effect of regeneration of healthy tissue i.e. tissue regeneration.

The composition and the formulation according to the present invention are also found to be effective in the treatment of diseases of the connective and glandular tissue such as abscesses and mastitis, and in the treatment of superficial diseases, for example in the treatment of skin eczema and psoriasis. In patients suffering from severe eczema and psoriasis on the hand palms, the application of the formulation for 15 minutes, 2 times per day leads to the complete disappearance of the disease after two days in the case of eczema, while in the case of psoriasis a net improvement after 5 days of treatment is shown.

Furthermore, the composition and the formulation according to the invention are found to be effective in the treatment of acute post-surgical inflammation, while ensuring an effect of stimulation of the processes of tissue regeneration, for example where a rapid functional recovery or rehabilitation through appropriate exercises is required to mobilize the party who suffered the surgical trauma.

Further advantages and characteristics of the composition and the formulation according to the present invention will become apparent to the skilled in the art from the following non-limiting examples of clinical studies of inflammatory diseases.

In the examples, as a parameter for the clinical evaluation of articular pathologies of the knee was used the KOOS index described in detail in the publications “ICRS Recommendation Document: Patient-Reported Outcome. Instruments for Use in Patients with Articular Cartilage Defects “Ewa M. Roos, et al. Cartilage, XX (X) 1-15 2011; “Measures of Knee Function” Natalie J. Collins et al. Arthritis Care & Research, Vol 63, No. S11, November 2011, pp. S208-S228, and “Development of the Italian version of the knee injury and osteoarthritis outcome score for patients with knee injuries: cross-cultural adaptation, dimensionality, reliability, and validity.” Monticone M, et al, Osteoarthritis Cartilage. 2012 April, 20 (4):330-5. Epub 2012 Jan. 10. With the required marginal adaptations, that index has also been used with regard to other districts such as the shoulder.

As a parameter for the evaluation of patients with rheumatoid arthritis was used the index RAOS described in “Validation of the Rheumatoid and Arthritis Outcome Score (RAOS) for the lower extremity.” Bremander A B, Petersson I F, Roos E M.; What Life Health Outcomes. 2003 Oct. 17; 1:55. For the evaluation of joint problems related to the district ankle and foot was used the index FAOS described in “Validation of the foot and ankle outcome score for ankle ligament reconstruction.” Roos E M, Brandsson S, Karlsson J., Foot Ankle Int 2001 October, 22 (10):788-94.

All publications mentioned above, related to the indices KOOS; RAOS and FAOS, are hereby incorporated by reference.

All patients in this study had suspended all other therapeutic treatment a month before the start of treatment cycles according to the present invention, unless otherwise specified.

The examples below refer to the figures, in which:

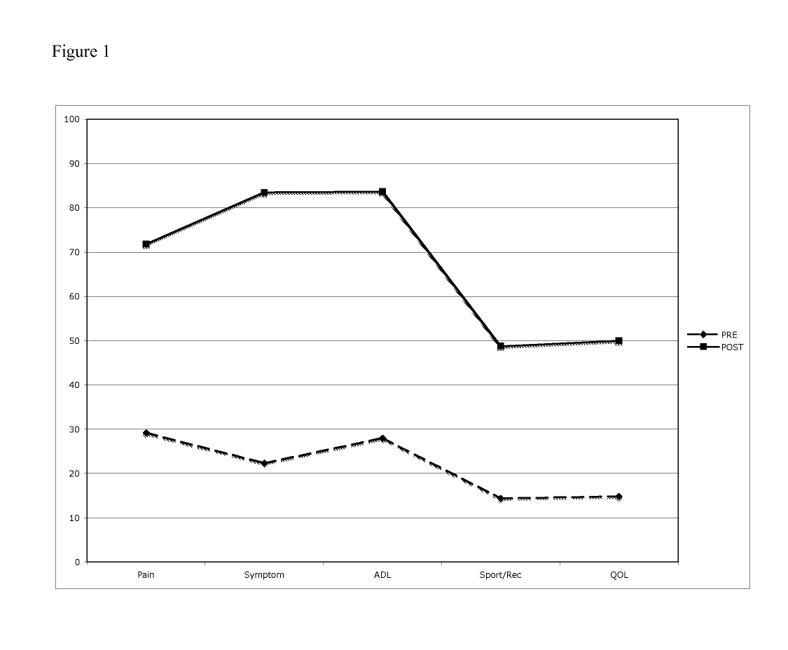

FIG. 1 shows a graph that represents the average values of the indices KOOS of eight patients suffering from chronic osteoarthritis at the anatomical district indicated under pathology in Table 1, before treatment according to example 1 (PRE) and after treatment (POST);

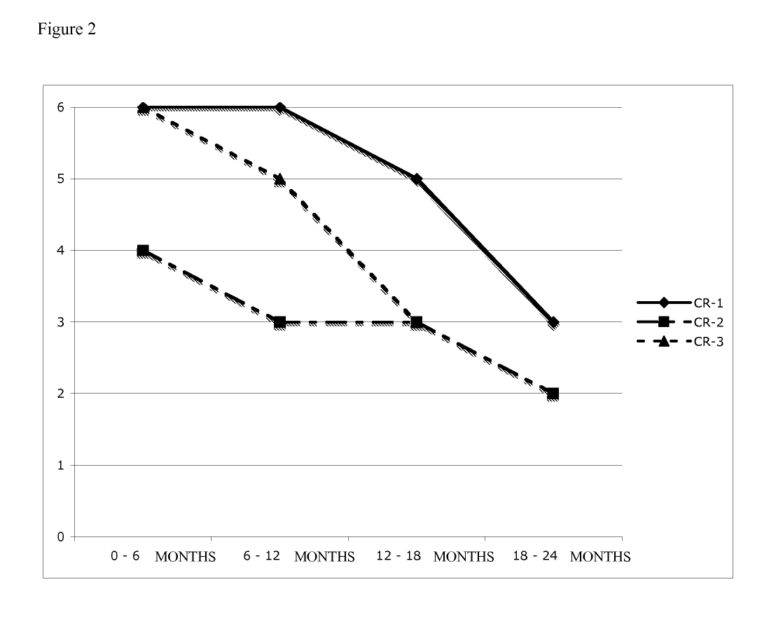

FIG. 2 shows a graph, which shows the frequency of cycles of treatments, seven days each, applied to patients of Example 1;

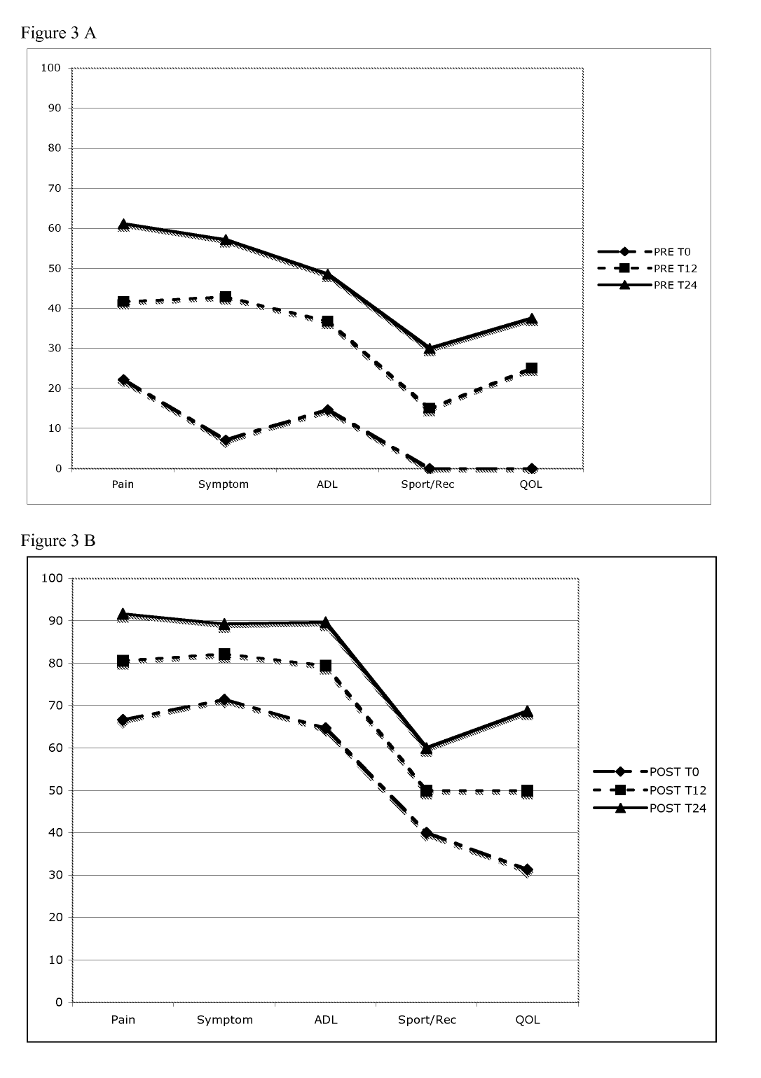

FIGS. 3A and 3B show graphs that show the values of the KOOS indices relating to the patient CR-1 of Example 1 before and after the various cycles of treatment;

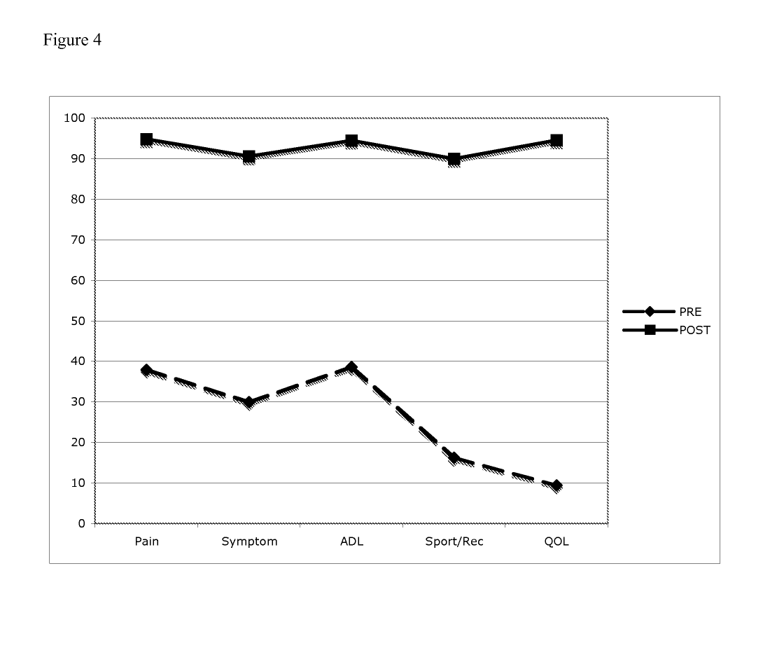

FIG. 4 shows a graph which shows average values of the KOOS indices of eight patients suffering from acute joint disease of the anatomical district indicated under pathology in Table 2, treated as described in Example 2;

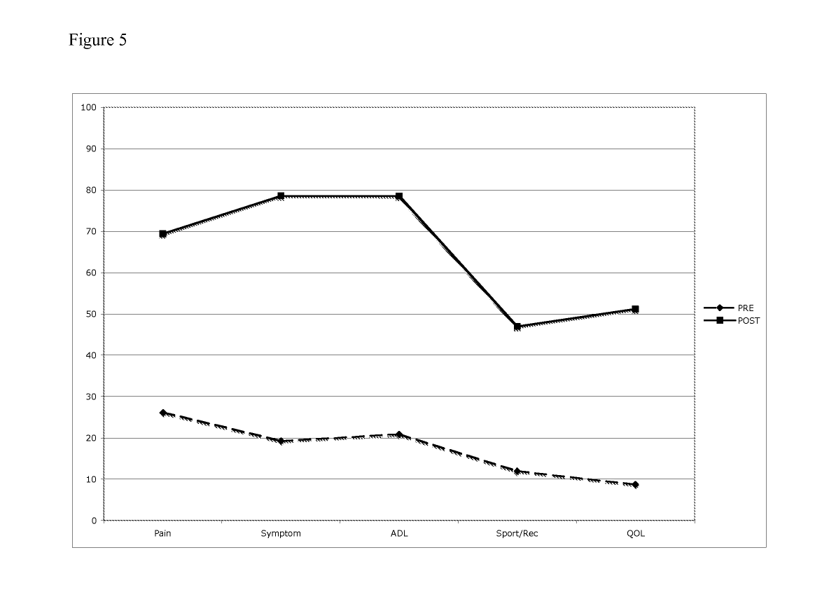

FIG. 5 shows a graph which shows average values of the RAOS indices of five patients suffering from chronic joint disease resulting from rheumatoid arthritis of the knee and ankle, treated as described in Example 3.

The results of treatment of the patients are shown in the figures according to the system of representation provided by the index KOOS for the knee and the FAOS for the district ankle-foot. For patients with shoulder pathology, the index KOOS FAOS has been adapted following the instructions received directly by the drafters of the index KOOS, FAOS and RAOS to ensure a uniform interpretation of the results. The reported results are the average of the measurements made by the patients included in the study.

In tables and figures, the values of the KOOS indices are expressed in cents, where 100 represents the complete well-being and 0 corresponds to a complete disability.

The indices related to pain, symptoms, activities in daily life (ADL), sport and recreation (Sport/Rec) and quality of life (QOL) are represented separately. The age of the patients, the duration of therapy in months, the number of cycles performed by each patient and the total number of treatment cycles are given in the tables.

Objective measurements by the physician, such as measurement of the circumference at the level of the knee for the evaluation of the edema-swelling reabsorption by applying accurate land marks, and objective assessments of patient mobility (ability of execution of specific movements) and radiologic assessment using magnetic resonance imaging, were also performed, when possible.

### EXAMPLE 1

### Treatment of Deep Chronic Inflammation with Tissue Damage and Degeneration

A formulation according to the present invention was prepared by suspending 34 g of dehydrated hen egg white in a mixture consisting of 24 g of propylene glycol, 24 g of pentaerythrityl tetraisostearate and 4 g of hectorite clay that were previously mixed.

The thus obtained composition was inserted in a bottle of the capacity of 300 ml which was then pressurized with 114 g of LPG 3.2 until reaching a pressure of 3-5 bar±0.5 bar at 20° C.

The thus obtained formulation A was then used in the treatment of 8 patients suffering from chronic inflammatory diseases joints, in particular osteoarthritis (OA), so distributed and shown schematically in Table 1:
6 patients suffering from chronic inflammatory diseases of the knee joint;
1 patient suffering from chronic inflammatory diseases of the shoulder joint;
1 patient suffering from chronic inflammatory diseases of the ankle joint.

In particular, formulation A was applied topically on the skin area affected by the inflammation, so as to form a continuous layer; a gauze of about 10×20 cm was soaked with about 20 ml of sterile water, and was then applied locally on the skin at the inflamed part, and covered with a thin waterproof film. The formulation was left in contact with the skin for a whole night. On awakening the waterproof film and gauze were removed and the treated area was rinsed with water. A cycle of treatment consists in applying the formulation for 7 consecutive nights. The cycles were repeated as often as indicated in Table 1.

The following Table 1 shows the characteristics of patients treated. The patients are indicated by the code CR-1 to 8.

TABLE 1
Patients with chronic OA

| | | Months of | Number of | |
|---|---|---|---|---|
| Patient | Age | therapy | treatment cycles | Pathology |
| cr-1 | 83 | 24 | 20 | knee OA |
| cr-2 | 63 | 24 | 12 | knee OA |
| cr-3 | 79 | 18 | 16 | knee OA |
| cr-4 | 85 | 8 | 6 | knee OA |
| cr-5 | 58 | 6 | 4 | knee OA |
| cr-6 | 55 | 4 | 3 | knee OA |
| cr-7 | 58 | 4 | 3 | ankle OA |
| cr-8 | 60 | 4 | 3 | shoulder OA |
| Average age | 67, 63 | | | |

| | |
|---|---|
| Total cycles of treatment | 67 |

All patients undergoing therapy had already been treated in previous years, starting from the onset of the disease, with conventional and unconventional therapeutic approaches, without success or with poor results. All patients, regardless of the therapeutic approach used, had noticed a progressive gradual worsening of symptoms, both considering clinical aspects and the indices of measurement of these aspects. Treatment approaches that had already been used without success or with poor results included the use of local and systemic anti-inflammatory drugs, local and systemic, NSAIDs, laser therapy, tecar therapy, tens, massage, steroid infiltrations, hyaluronates, non-conventional compounds such as mesotherapy with various preparations, local application of herbs derivatives, clay application, mud baths, thermal treatments in addition to using the latest biotechnology drugs recently introduced on the market (such as monoclonal antibodies). At the time of start of therapy, three of the eight patients were candidates for intervention for the insertion of a prosthetic knee.

Results

The evaluation of the results was recorded for the entire duration of the applications.

In FIG. 1, the dashed line represents the clinical condition before treatment (PRE) of the duration of 7 days, while the continuous line represents the clinical condition after the treatment cycle (POST).

As it is apparent from FIG. 1, by analyzing the trend of the KOOS indices before and after the cycle of treatment of 7 days with the formulation according to the invention, a marked improvement of all indices is evident, both from an overall and a separate evaluation.

All patients suffering from chronic inflammatory processes that were subjected to the treatment reported a marked clinical improvement, with marked reduction of symptoms, edema, stiffness, pain. Also, an increase in the mobility of the treated joint was shown, resulting in noticeable improvement in the quality of life. Improved mobility comprised both the mobility of the isolated joint and that of the daily exercises.

In particular, the most immediate effect was the reduction of stiffness, joint edema or swelling and pain, which was visible already after the first application. The measurement of the circumference of the joint, before and after the applications, showed a marked and significant reduction of the edema.

The pain reduction was clear during the first two or three applications and it was evident both in a static position, that is, with an immobile limb, and in motion.

All patients, after each cycle of treatment of 7 days stated that they had achieved greatly superior clinical improvements with respect to any other previously tested therapy. All patients recovered clinical skills that were lost months or years before. The majority of patients were able to practice again activities that had previously gradually excluded from everyday life (walking, cycling etc.) and reported a marked improvement, with final disappearance of the pain, both in a static position and at awakening.

All patients reported a feeling of softness and elasticity at the skin surface where the inventive formulation was applied, that is indicative of a tissue regeneration.

For patients shown in Table 1 with the abbreviations CR-1, CR-2 and CR-3, the treatment was repeated periodically, when symptoms reappeared, for periods of 24 or 18 months. The clinical observation of the inflammatory state course revealed a progressive improvement of the clinical condition that could be measured in time unit. In fact, it was observed a gradual lengthening of the period of effectiveness of the single treatment cycle that corresponds to a progressive lower frequency of the therapy cycles. Furthermore, a reduction in intensity of the inflammatory state was observed when symptoms reappeared, indicative of a substantial reversal of degenerative pathology.

FIG. 2 shows that in the first months of therapy, patient CR-1 required each month the repetition of the treatment cycle (6 cycles over a period of 6 months), while at the end of the period of two years of continuation of cycles of therapy, the same patient required 3 cycles of therapy every 6 months. The same effect is also visible in patient CR-3, who passed from a request of frequency of six cycles of therapy every six months, to three cycles every 6 months after 18 months of therapy. The patient CR-2 at the beginning of therapy required 4 cycles of therapy every 6 months, while at the end of the period of 24 months of therapy he required 2 cycles every 6 months.

FIGS. 3A and B show indexes KOOS of patient CR-1 suffering from a highly disabling knee osteoarthritis, for whom insertion of prosthetic joint had been recommended at the time of beginning of the clinical study. FIG. 3A shows the indices KOOS of the CR-1 patient detected before treatment (PRE TO), before a treatment cycle at the twelfth month of therapy (PRE T12) and before treatment cycle at the twenty-fourth month therapy (PRE T24). FIG. 3B shows the indices for the same patient after the first therapy treatment cycle (POST TO), after a treatment cycle at the twelfth month of therapy (POST T12) and after a treatment cycle at the twenty-fourth month of therapy (POST T24).

FIG. 3A shows that at the beginning of the study the patient was severely compromised in all the five indices analyzed. Already after 12 months and even more clearly after 24 months of regular applications, which, however, became less and less frequent with the passage of time, the patient appeared in markedly improved clinical condition. FIG. 3B shows that the treatment effect, already marked at the beginning, became increasingly evident after 12 and 24 months. Furthermore, even if the duration of a cycle of treatment was maintained at 7 days, the patient reported well-being already after two or three applications (or days of treatment). The significance of this clinical trend is explainable not only as the result of an anti-inflammatory activity, but as result of the simultaneous true tissue regeneration activity.

Therefore, the composition according to the invention, even after several cycles of treatment, does not involve addiction or dependence. In contrast, the anti-inflammatory activity tends to decrease the severity of the disease due to the activity of regeneration of damaged tissue.

### EXAMPLE 2

### Acute Inflammation and Deep Tissue Damage

A formulation C was prepared by mixing dehydrated hen egg albumen, glycerin, sodium alginate and water. The amount of dehydrated albumen was equal to 30% by weight compared to the total of the formulation. Formulation C was coated on substrates of non-woven fabric of 50 g/m² and 10 cm×10 cm dimensions, in such quantity as to obtain a quantity of dehydrated egg albumen on the support of approximately 6 mg/cm². After drying of the formulation, reeling, cutting the reel in strips and die cutting, the patches were packaged as single-dose patch.

The patches were used in the treatment of 8 patients with a mean age of 46.13 years (range of 35-62 years) suffering from acute inflammatory joint diseases so divided and distributed as schematically shown in Table 2:
4 patients were suffering from inflammatory disease of the knee joint, including: 1) an acute posterior osteoarthritis of the knee with bursitis and obvious bone marrow edema of the femur, that was revealed by magnetic resonance imaging, and 2) an acute osteoarthritis with patellar bursitis, and 3) a cyst of Baker, 4) a pes anserine bursitis or knee medial dystrophic periarthritis;
1 suffering from inflammatory disease of the shoulder joint (traumatic injury to the rotator cuff and the long head of the biceps);
1 suffering from inflammatory disease of the ankle joint (distortion);
1 suffering from acute inflammatory joint disease of the elbow (epicondylitis);
1 suffering from inflammatory disease of the Achilles tendon (tendonitis).

The patches were made wet before use and applied on the skin at the inflamed part, and left in contact with the skin for a whole night. Upon awakening the patch was removed and the treated area was rinsed with water. A cycle of treatment consists of applying the patches for 7 consecutive nights.

The following Table 2 shows the characteristics of patients which are indicated with the code AC-1 to 8.

TABLE 2
Acute patients

| | | Months of | Number of cycles | |
|---|---|---|---|---|
| Patient | Age | therapy | of treatment | Pathology |
| AC-1 | 62 | 2 | 4 | Knee 1) |
| AC-2 | 47 | 1 | 2 | Knee 2) |
| AC-3 | 42 | 1 | 1 | Ankle |
| AC-4 | 48 | 1 | 2 | Knee 3) |
| AC-5 | 51 | 1 | 1 | Shoulder |
| AC-6 | 41 | 1 | 2 | Knee 4) |
| AC-7 | 35 | 1 | 1 | Elbow |
| | | | | epicondylitis |
| AC-8 | 43 | 1 | 1 | Achilles tendon |
| Average age | 46, 13 | | | |
| Age range | 35-62 | | | |

| | |
|---|---|
| Total cycles of treatment | 14 |

Some of the patients included in the study (AC-1, AC-2 AC-4, AC-5, AC-8) and treated had already been subjected in previous months to conventional and non-conventional therapeutic approaches, without success or with poor results. In particular, had been subjected to other treatments: an acute posterior osteoarthritis of the knee with bursitis and obvious bone marrow edema of the femur shown by magnetic resonance; an acute osteoarthritis with patellar bursitis, a pes anserine bursitis or knee medial dystrophic periarthritis, a patient suffering from inflammatory disease of the shoulder joint (traumatic injury to the rotator cuff and the long head of the biceps); a patient suffering from inflammatory disease of the Achilles tendon (acute tendonitis of the Achilles tendon).

Treatment approaches that had already been used without success or with poor results included the use of local and systemic anti-inflammatory drugs, NSAIDs local and systemic, laser therapy, tecar, tens, massage, steroid infiltrations, hyaluronates, non-conventional compounds such as mesotherapy with various preparations, local application of derivatives of herbs, clay application, mud baths and thermal treatments.

Parameters related to symptoms such as swelling, rattles, blocks, extension and bending; stiffness, pain, function and mobility of the joint in daily activities and sports/recreational activities, impact on quality of life were evaluated.

Results

The evaluation of the results was recorded for the entire duration of the applications; patients suffering from acute illnesses were followed for a period ranging from 2 months to less than 1 month, as shown in table 2.

The results obtained are shown in FIG. 4, in which the dotted line refers to the evaluations carried out before treatment (PRE), while the continuous line refers to the evaluations performed subsequently to the cycle of treatment (POST).

By analyzing the performance of the KOOS indices before and after the treatment cycle of 7 days with the composition according to the invention, a marked improvement of all indices with reestablishing a clinical level corresponding to total healing is evident. The healing times were very short when compared with the periods necessary for the commonly used standard therapies. In the five cases, where the patient had been previously treated with conventional and non-conventional therapies but without success, a complete recovery of patients was observed. In cases where more treatment cycles were necessary, the cycles were separated by one or more days of interruption or multiple cycles were applied without interruption.

More in detail, all of the treated subjects reported a clear improvement of the clinical conditions as a result of the treatment, in particular, a rapid and marked reduction in edema and pain with a quick recovery of joint function was observed.

### EXAMPLE 3

### Treatment of Chronic Inflammation Deep Rheumatoid Arthritis

A formulation as obtained in Example 1 was prepared and used in the treatment of 5 patients with a mean age of 70.80 years, range 48 to 84 years old, suffering from rheumatoid arthritis with involvement of the joints of the knee and ankle.

The formulation was applied topically on the skin at the inflamed part. The application was left in contact with the skin for a whole night. Upon awakening waterproof film and gauze were removed and the treated area was rinsed with water. A cycle of treatment consists of applying the formulation for 7 consecutive nights. The cycles were repeated in the cases mentioned below for a total period up to 14 months, with the frequency specified in the following Table 3, in which patients are indicated with an RA-1 to 5.

TABLE 3
Patients chronic RA
Months of therapy Patient Age Number of treatment cycles

| | | Months of | Number of |
|---|---|---|---|
| Patient | Age | therapy | treatment cycles |
| RA-1 | 82 | 14 | 12 |
| RA-2 | 62 | 6 | 5 |
| RA-3 | 78 | 5 | 4 |
| RA-4 | 84 | 4 | 3 |
| RA-5 | 48 | 4 | 2 |
| Average age | 70, 80 | | |
| Age range | 48-84 | | |

| | |
|---|---|
| Total treatment cycles | 26 |

For these patients, the result of a total of 26 treatment cycles, each lasting 7 days, for a treatment period varying from 14 months to 4 months depending on the conditions of the various initial clinical patients, was rated.

All patients undergoing treatment had already been subjected in previous years or from the beginning of the disease, to conventional and unconventional therapeutic approaches without success or with poor results. In all patients, regardless of the therapeutic approach used, the patient had noted a gradual worsening of symptoms over time characterized by a worsening of clinical conditions and of the indices of measurement thereof. Treatment approaches that had already been used without success or with poor results included the use of local and systemic anti-inflammatory drugs, local and systemic NSAIDs, laser therapy, tecar, tens, massage, steroid infiltrations, hyaluronates, non-conventional compounds such as mesotherapy with various preparations, local application of derivatives of herbs, clay application, mud baths, thermal treatments in addition to using the latest biotechnology drugs recently introduced on the market (such as monoclonal antibodies).

Clinical parameters were evaluated using the RAOS index and the FAOS index. From the analysis of the questionnaires it was showed that the RAOS and FAOS index, are substantially superimposable, so the data for the patients suffering from rheumatoid arthritis are all expressed with the RAOS index that refers to the underlying disease and not to the anatomical region.

Results

The evaluation of the results was recorded for the entire duration of the applications; patients suffering from rheumatoid arthritis were followed for a period ranging from 14 months to 4 months as shown in Table 3.

The results are shown in FIG. 5 in which the dashed line represents the clinical condition before treatment (PRE), the continuous line represents the clinical condition after the cycle of treatment (POST).

As it appears from the figure, by analyzing the trend of the RAOS indices before and after the treatment cycle of 7 days with the composition according to the invention, a marked improvement of all indexes is obtained, both when considering the 5 indexes together and individually. The detection of greater improvement the components pain, symptoms, activities of daily living (ADL), compared to a more modest improvement observed in the components of sports and recreation (Sport/Rec), quality of life (QOL), is typical of patients with worsening chronic pathologies. Moreover, this difference is more marked in chronic patients in more advanced phase of disease in which the joint in question has characteristics of advanced tissue degeneration. Even in patients with rheumatoid arthritis, treated for a long time, there has been a gradual improvement in symptoms by means of repeated cycles of treatment and there is a substantial reversal of the degenerative process of the tissues (articular tissue in rheumatoid diseases) and a gradual tissue regeneration that is clearly observable from the clinical point of view.

### EXAMPLE 4

### Treatment of Cutaneous Inflammation

A formulation B according to the invention, obtained by mixing 15 g of dehydrated egg white and 85 g of shea butter, was used in treatment of a patient suffering from eczema of the hands and of a patient suffering from psoriasis localized to the elbows.

The patient with eczema was suffering from a severe form of contact eczema of the hands as a result of an already known hypersensitivity to a component of detergents. The hands, before treatment, appeared flushed with areas of abrasion and presence of areas of desquamation and scabs. These phenomena were present with particular intensity on both the palms of the hands and fingers. In similar cases, which were already visible on other occasions, the patient was treated with the ointments containing cortisone, emollient creams and moisturizers. In previous episodes using the above-mentioned therapy, the disease was resolved in variable times, depending on the severity of symptoms, after a treatment of variable duration from a minimum of two weeks to four weeks. In the present case, the patient had got eczema three days before and had not yet started the usual and described above therapy. Formulation B was applied to the patient, two times per day. After two days of treatment, the patient was completely healed and the skin of the hands was healthy and integer with complete disappearance of redness, desquamation and scabs.

The effect of the composition was therefore tested on a patient suffering from psoriasis vulgaris (or plaque or nummular psoriasis) to the elbows characterized by localized areas of increased epidermal proliferation, formation of silvery or micaceous scales that covered erythematous plaques. The patient was 82 years of age and had been suffering from psoriasis for about twenty years and was subject to periods of relative stability followed by periods of exacerbation of the disease. For the containment of the disease the patient frequently used, among other, emollient ointments, keratolytic ointments, vitamin A and D derivatives, and corticosteroids: the effects of these therapies were not evident and measurable in terms of real disease reduction.

Formulation B was applied to the patient two times per day. After five days of treatment, corresponding to ten applications, the part affected by the disease was markedly improved with marked reduction in the number of scales and intensity and extent of the erythematous areas.

In light of the obtained results on the skin affected by chronic or recurrent pathologies it is evident that, also at the skin, the composition according to the invention has both an anti-inflammatory effect and an effect of real regeneration of the skin and skin appendages. The composition according to the invention is therefore also suitable for the therapy of inflammatory diseases of the skin (such as acne, rosacea etc.) and psoriasis. It can also be used for cosmetic purposes on healthy skin with regenerative purpose.

### EXAMPLE 5

### Treatment of Subareolar Abscess or Mastitis

Patches according to the invention obtained as in Example 2 were used in the treatment of a patient suffering from subareolar abscess or mastitis. The 42 years old female patient had a typical subareolar abscess in the form of relapse from a previous abscess that was treated two months before with therapy of 10 days duration including oral antibiotics, NSAIDs, cortisone and antibiotic ointment for topical use.

The relapse was characterized by redness, swelling and subareoalar pain. The patient was treated with topical application of the patches, i.e. on the breast surface, with nightly application of about 10 hours repeated for 5 times. Already after the first application, the part affected by the abscess appeared decidedly improved at removal of the patch, with almost complete disappearance of the swelling, redness and complete pain relief. After the second application, the part was completely healed with total disappearance of the inflammation signs. The treatments were continued until completion of the 5-day schedule. No other drugs such as antibiotics, local or systemic corticosteroids and NSAIDs. were used for the treatment of the patient with suberaolar abscess.

### EXAMPLE 6

### Treatment of Puerperal Mastitis

Formulation D was prepared by mixing dehydrated hen's egg white, in an amount equal to 30% by weight, petrolatum jelly in an amount of 57.65% by weight, petrolatum oil in an amount of 11.94% by weight, and vitamin E acetate in an amount of 0.41% by weight.

Formulation D was used in the treatment of a patient with puerperal mastitis.

The 31 years old female patient after childbirth, during the third month of breastfeeding, showed a typical mastitis in the form of relapse from the previous two episodes occurred two and four weeks before, treated with therapies of 10 days each including oral antibiotics, NSAIDs, cortisone and antibiotic ointment for topical use. Also hormone therapy to reduce the production of milk was introduced during the second treatment.

The relapse was characterized by redness, swelling and pain.

The patient was treated with application of formulation D topically, i.e. on the surface of the breast, covered with a gauze wet with water. The gauze was kept overnight for about 8 hours and repeated for 5 nights. Already after the first application the area affected by the abscess appeared decidedly improved at removal of the gauze, with the almost complete disappearance of the swelling, redness and complete pain relief. After the second application, the part was completely healed with total disappearance of the signs of inflammation. No other drugs such as antibiotics, local or systemic corticosteroids and NSAIDs were used for the treatment of the patient with suberaolar abscess.

A further relapse of mastitis after three weeks was treated and resolved as described above using three applications of formulation D for 8 hours during the night for three consecutive nights.

1.-19. (canceled)

20. A method of treating inflammatory diseases associated with tissue degeneration and/or tissue damage in humans or animals, comprising topically administering an effective amount of dehydrated avian egg albumen, or a derivative or extract thereof to an area of the skin of a human or animal requiring such treatment.

21. The method of claim 20, wherein avian egg albumen derivative or extract thereof is part of a topical composition and said composition further comprises a substance selected from the group consisting of NaCl, antibiotics, antimycotics, antioxidants, steroidal and nonsteroidal antinflammatories, myorelaxants, molecules having proteolytic activity, molecules having anticoagulant activity, biotechnology drugs and drugs of natural origin.

22. The method of claim 20, wherein the inflammatory disease is a chronic degenerative inflammatory disease.

23. The method of claim 20, wherein the inflammatory disease is associated with tissue degeneration and/or tissue damage due to autoimmune diseases.

24. The method of claim 20, wherein the inflammatory disease is osteoarthrosis, osteoarthritis or rheumatoid arthritis.

25. The method of claim 20, wherein the inflammatory disease is tissue damage of the skin or mastitis.

26. The method of claim 20, wherein the inflammatory disease includes parenchymatous tissues.

27. The method of claim 20, wherein the inflammatory disease is of viral etiology or fungal etiology.

28. The method of claim 20, wherein the inflammatory disease is associated with tissue degeneration and/or tissue damage after stress and/or functional recovery after stress.

29. The method of claim 20, wherein the dehydrated avian egg albumen, derivative or extract thereof is part of a formulation in the form of a cream, gel, ointment, spray, medicated gauze, oil in water emulsion or water in oil emulsion, suspension, poultice, medicated implant, micro-emulsion and nano-emulsion, two-phase micellar gel, bath foam, medicated patch or pre-impregnated patch, or lotion.

30. The method of claim 29, wherein the formulation further comprises an excipient selected from the group consisting of shea butter, ethylene glycol, diethylene glycol, triethylene glycol, propylene glycol, glycerol, vaseline oil, and sodium alginate.

31. The method of claim 29, wherein the formulation comprises dehydrated hen egg albumen, propylene glycol, pentaerythrityl tetraisostearate and hectorite clay.

32. The method of claim 29, wherein the formulation is administered via a patch having a support impregnated with said formulation.

33. The method of claim 29, wherein the formulation is administered via a spray bottle containing the formulation and a pressurized aliphatic hydrocarbon.

34. The method of claim 29, wherein the formulation is in the form of a cream comprising dehydrated hen egg albumen in an amount of between 20% and 40% by weight, petrolatum in an amount of between 60% and 80% by weight, and vitamin E acetate in an amount of between 0.1% and 1% by weight.

35. The method of claim 34, wherein the cream comprises
a) between 25% and 35% by weight dehydrated hen egg albumen,
b) between 65% and 75% by weight petrolatum, and
c) between 0.3% and 0.5% by weight of vitamin E acetate.

36. The method of claim 29, wherein the formulation comprises
a) 5% to 40% by weight of dehydrated avian egg albumen; and
b) from 60% to 95% by weight of water.

37. The method of claim 36, wherein the formulation comprises
a) from 20% to 30% by weight; dehydrated avian egg albumen and
b) from 70% to 80% by weight of water.
